(19) United States
(12) Patent Application Publication
(10) Pub. No.: US 2026/0058011 A1
(43) Pub. Date: Feb. 26, 2026

(54) ARTICLES AND METHODS FOR ARTIFICIAL INTELLIGENCE DRIVEN TRACKING OF THE PROGRESSION OF PRE-CLINICAL AMYLOID CARDIOMYOPATHY

(71) Applicant: YALE UNIVERSITY, New Haven, CT (US)

(72) Inventors: Rohan Khera, New Haven, CT (US); Evangelos Oikonomou, New Haven, CT (US)

(21) Appl. No.: 18/813,882

(22) Filed: Aug. 23, 2024

(51) Int. Cl. G16H 50/20; A61B 5/318; A61B 8/08; G16H 10/60

(52) U.S. Cl. CPC G16H 50/20; A61B 8/0883; A61B 8/5207; G16H 10/60; A61B 5/318

(57) ABSTRACT

Provided herein is a method of training a machine-learning model to detect cardiomyopathy in a subject, the method including providing a training dataset, the training dataset including cardiac diagnostic data from a group of subjects; identifying, in the training dataset, cardiac diagnostic data from positive subjects with cardiomyopathy and cardiac diagnostic data from control subject without cardiomyopathy; and training the machine-learning model with the training dataset to discriminate between the cardiac diagnostic data from positive subjects and the cardiac diagnostic data from control subjects. Also provided herein are a method of detecting cardiomyopathy in a subject using the machine-learning model trained according to the methods disclosed herein, an apparatus for implementing the method of detection cardiomyopathy, and a computer readable storage medium storing computer-executable instructions for performing the method.

### BACKGROUND

Cardiomyopathies, diseases of the heart muscle, represent a highly heterogeneous groups of disorders, which often follow an insidious course before resulting in clinical symptoms. One example is amyloid cardiomyopathy, with various subtypes such as transthyretin amyloid cardiomyopathy (ATTR-CM) and light chain amyloid cardiomyopathy (AL-CM), a progressive and life-threatening disease that remains largely under-recognized, under-diagnosed, and under-treated. There are subtypes that are highly treatable with emerging therapeutics. For example, a novel group of therapies can effectively modify clinical disease progression in ATTR-CM thus improving overall outcomes and survival. These treatments stabilize disease, but often are started too late. Indeed, ATTR-CM generally follows a rapid progression course following the onset of symptoms (median of 2-6 years of survival in ATTR-CM).

Accordingly, there remains a need in the art for articles and methods that improve upon existing articles and methods for detecting cardiomyopathies and determining the appropriate treatment early during the pre-clinical stages of the disease. The present disclosure meets this need.

### SUMMARY

In one aspect, provided herein is a method of training a machine-learning model to detect cardiomyopathy in a subject, the method including providing a training dataset, the training dataset including cardiac diagnostic data from a group of subjects; identifying, in the training dataset, cardiac diagnostic data from positive subjects with cardiomyopathy and cardiac diagnostic data from control subject without cardiomyopathy; and training the machine-learning model with the training dataset to discriminate between the cardiac diagnostic data from positive subjects and the cardiac diagnostic data from control subjects. In some embodiments, the control subjects are at least one of age-matched to the positive subjects, sex-matched to the positive subjects, matching based on clinical and biological measurements of cardiac remodeling, and a combination thereof.

In some embodiments, the cardiac diagnostic data includes electrocardiographic (ECG) signals, images of ECGs, echocardiographic imaging, cardiac magnetic resonance imaging, nuclear cardiology examinations, or a combination thereof. In some embodiments, the method further includes pre-processing the echocardiographic imaging, the pre-processing of the echocardiographic imaging including loading pixel data; feeding randomly sampled images and/or frames through a convolutional neural network (CNN), the CNN classifying the frames by assigning a probability that a given video corresponds to a standard anatomical view; assigning a predicted view according to the highest assigned probability; cleaning and de-identifying the frames; augmenting the data; training a binary, video-level classifier to detect the presence of cardiomyopathy from controls; and normalizing intensities of each video clip.

In some embodiments, following the training step, the machine-learning model is configured to identify key patterns of cardiomyopathy. In some embodiments, following the training step, the machine-learning model is configured to track longitudinal changes in the probability of cardiomyopathy among patients. In some embodiments, following the training step, the machine-learning model is configured to detect subclinical cardiomyopathy. In some embodiments, following the training step, the machine-learning model is configured to output a probability of a subject developing cardiomyopathy.

In some embodiments, the cardiomyopathy includes transthyretin amyloid cardiomyopathy (ATTR-CM). In some embodiments, the subject was determined to be positive for ATTR-CM through an abnormal bone scintigraphy study or cardiac magnetic resonance imaging.

Also provided herein is a method of detecting cardiomyopathy in a subject, the method including providing cardiac diagnostic data from the subject; and inputting the cardiac diagnostic data from the subject to the machine-learning model trained according to any of the methods disclosed herein; wherein the machine-learning model outputs a probability of the subject developing cardiomyopathy based upon the cardiac diagnostic data. In some embodiments, the ECG data is unimodal. In some embodiments, the ECG data is multimodal.

In some embodiments, the cardiomyopathy includes restrictive or infiltrative cardiomyopathies with long indolent preclinical course, or combinations thereof. In some embodiments, the cardiomyopathies with long indolent preclinical course include amyloid cardiomyopathy, hypertrophic cardiomyopathy, sarcoid cardiomyopathy, or a combination thereof.

In some embodiments, the method further includes determining that the subject has cardiomyopathy based upon the output from the machine-learning model. In some embodiments, the cardiomyopathy is pre-clinical cardiomyopathy. In some embodiments, the method further includes guiding the subject's eligibility for risk modifying therapies to reduce their risk of progression. In some embodiments, the method further includes tracking a response to disease-modifying therapies in progressive cardiomyopathies using output probabilities of the model. In some embodiments, the method further includes determining the subject's eligibility for use of a disease-modifying therapy or inclusion in a clinical study or clinical trial using output probabilities of the model.

Also provided herein is an apparatus for detecting cardiomyopathy in a subject, the apparatus including a processor; a memory unit; and a communication interface; wherein the processor is connected to the memory unit and the communication interface; and wherein the processor and memory are configured to implement the method according to any of the embodiments disclosed herein.

Also provided herein is a computer readable storage medium storing computer-executable instructions for performing the method according to any of the embodiments disclosed herein.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 shows a schematic illustrating a study overview according to an embodiment of the disclosure. Deep learning algorithms to discriminate bone scintigraphy-positive cases of ATTR-CM from age-and sex-matched controls using standard TTE videos or ECG images. These were subsequently deployed across independent sets of patients with longitudinal monitoring by TTE or ECG pre-dating their referral for bone scintigraphy testing. This overall objective was to examine the ability of the AI models to detect changes in TTE or ECG signatures that precede clinical disease and diagnosis. Such AI-enabled TTE or ECG signatures may be used to forecast the development of ATTR-CM, thus offering a standardized and scalable platform for longitudinal monitoring and screening in the community. AI: artificial intelligence; ATTR-CM: transthyretin amyloid cardiomyopathy; ECG: electrocardiography; Tc99m-PYP: pyrophosphate (bone scintigraphy tracer); TTE: transthoracic echocardiography.

FIG. 2 shows a schematic illustrating a summary of a study dataset according to an embodiment of the disclosure. AI-models were trained on transthoracic echocardiograms (TTE) and 12-lead electrocardiographic (ECG) images from patients with ATTR-CM (based on a positive bone scintigraphy study done within 12 months, or anytime in the past) as well as age-and sex-matched controls across the Yale-New Haven Health System (YNHHS). Models were subsequently deployed across independent sets of patients in YNHHS who had sequential TTE studies, or ECGs performed in the years leading up to confirmatory testing by bone scintigraphy. This design evaluated the progression of AI-Echo or AI-ECG probabilities as non-invasive markers of sub-clinical ATTR-CM progression. AI: artificial intelligence; ATTR-CM: transthyretin amyloid cardiomyopathy; ECG: electrocardiography; Tc99m-PYP: pyrophosphate (bone scintigraphy tracer); TTE: transthoracic echocardiography; YNHHS: Yale-New Haven Health System.

FIGS. 3A-D show graphs illustrating longitudinal changes in AI-Echo and AI-ECG ATTR-CM probabilities in the YNHHS population stratified by bone scintigraphy positivity. The panels illustrate the mean (with error bars denoting the 95% confidence interval of mean) of the AI-Echo (A, B) and AI-ECG-derived probabilities (C, D) across patients who went on to have a positive (orange color) vs negative (blue color) bone scintigraphy study. The x axis denotes the time between the TTE/ECG and the timing of the bone scintigraphy study, summarized across discrete time groups (negative time differences suggest that the TTE/ECG was performed before bone scintigraphy). The brackets below each period along the x axis denote the number of positive and negative studies or patients. Results are presented both at the study-level (A, C), as well as at a participant level (B, D) by taking the chronologically latest prediction for each unique individual in each period. ATTR-CM: transthyretin amyloid cardiomyopathy; ECG: electrocardiography; TTE: transthoracic echocardiography; YNHHS: Yale-New Haven Health System.

FIG. 4 shows a graph illustrating an association between AI-Echo and AI-ECG probability of ATTR-CM and all-cause mortality among participants with negative bone scintigraphy. The graphs denote the association (hazard ratio, with corresponding 95% confidence interval) for each independent predictor (AI-Echo in blue, AI-ECG in red) and all-cause mortality, as derived from Cox regression models adjusted for age and sex. Curves were fitted using restricted cubic splines for AI-Echo and AI-ECG probabilities. AI: artificial intelligence; ATTR-CM: transthyretin amyloid cardiomyopathy; ECG: electrocardiography.

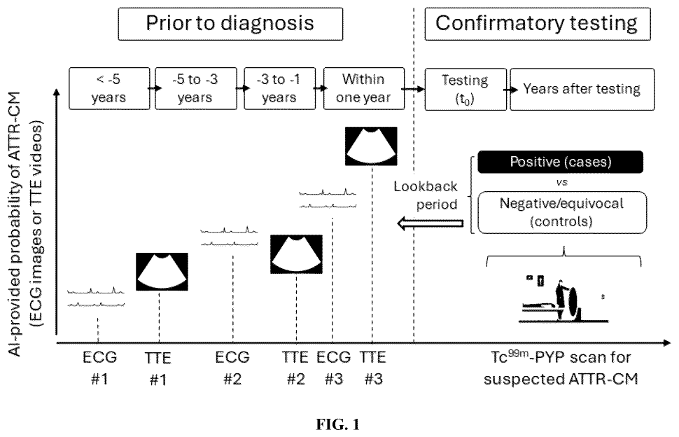

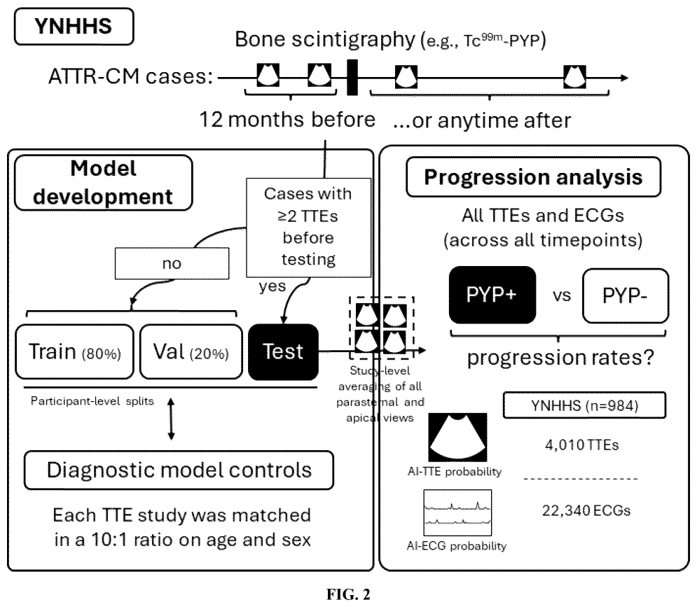

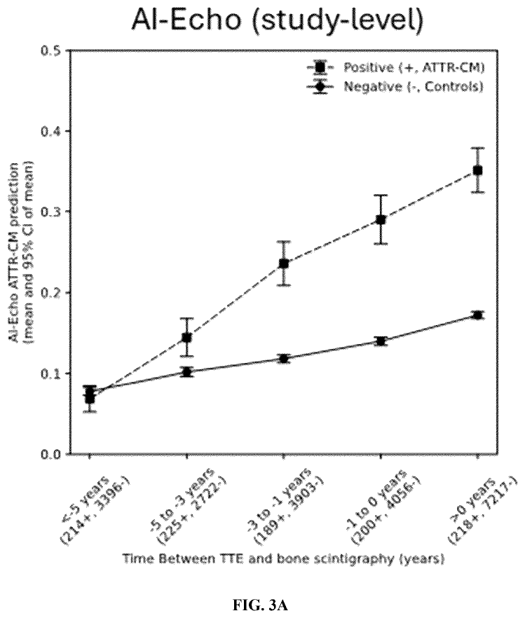

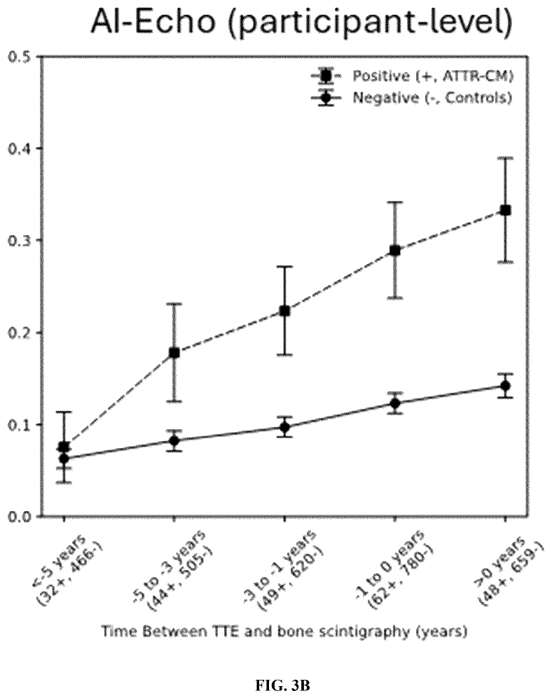

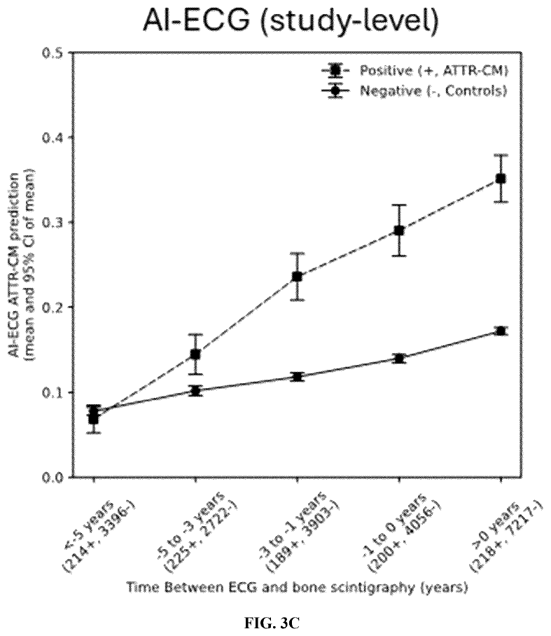

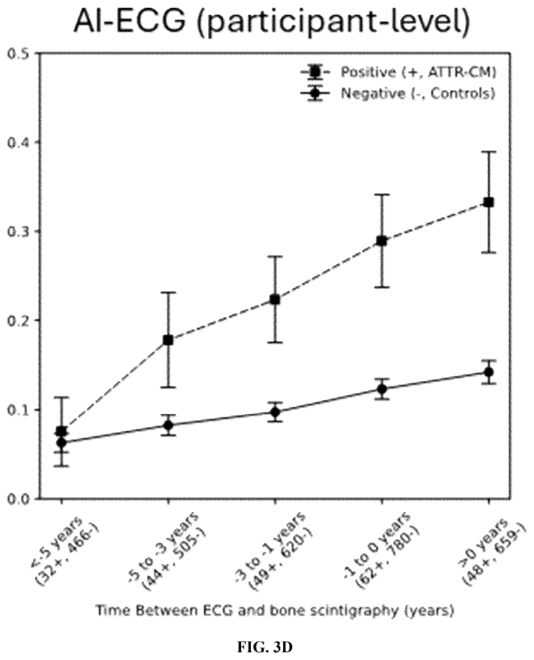

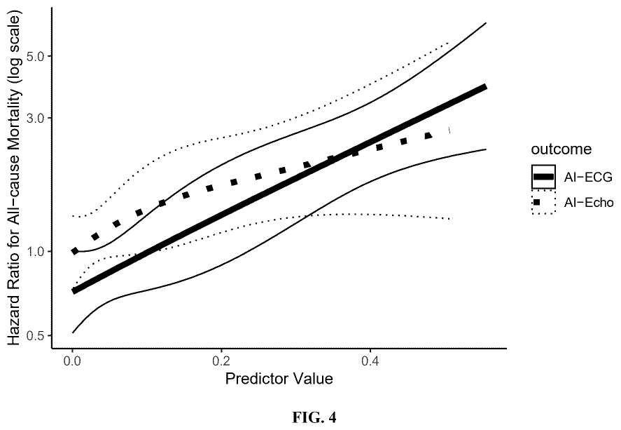

### DETAILED DESCRIPTION

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in molecular biology, immunology, animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

As used herein, the term “ratio” refers to a relationship between two numbers (e.g., scores, summations, and the like). Although, ratios can be expressed in a particular order (e.g., a to b or a: b), one of ordinary skill in the art will recognize that the underlying relationship between the numbers can be expressed in any order without losing the significance of the underlying relationship, although observation and correlation of trends based on the ration may need to be reversed. For example, if the values of a over time are (4, 10) and the values of b over time are (2, 4), the ratio a: b will equal (2, 2.5), while the ratio b:a will be (0.5, 0.4). Although the values of a and b are the same in both ratios, the ratios a:b and b:a are inverse and increase and decrease, respectively, over the time period.

### DETAILED DESCRIPTION

Provided herein are methods of training a machine-learning model to detect cardiomyopathy in a subject. In some embodiments, the method includes providing a training dataset, the training dataset including cardiac diagnostic data, such as electrocardiographic (ECG) data, transthoracic echocardiography (TTE) data, or a combination thereof from a group of subjects; identifying, in the training dataset, cardiac diagnostic data from positive subjects with cardiomyopathy and from control subjects without cardiomyopathy; and training the machine-learning model with the training dataset to discriminate between the cardiac diagnostic data from positive subjects and the cardiac diagnostic data from control subjects. In some embodiments, the control subjects are at least one of age-matched to the positive subjects, sex-matched to the positive subjects, matched for standard clinical metrics (e.g., left ventricular thickness), or a combination thereof.

Suitable cardiomyopathies include, but are not limited to, amyloid cardiomyopathy, including transthyretin amyloid cardiomyopathy (ATTR-CM), or any other inherited or acquired cardiomyopathy with a progressive course. In some embodiments, the cardiac diagnostic data from positive subjects includes data taken within a specified time window before (i.e., one year) and/or anytime after the subject was determined to be positive for cardiomyopathy. As will be appreciated by those skilled in the art, determining that a subject is positive for cardiomyopathy will depend upon the specific type of cardiomyopathy. For example, a subject may be determined to be positive for ATTR-CM through an abnormal bone scintigraphy study, cardiac magnetic resonance imaging, biopsy, or any combination thereof.

The cardiac diagnostic data in the training dataset includes any data suitable for detecting changes in a subjects cardio vasculature that relate to or are impacted by a cardiomyopathy of interest. Suitable cardiac diagnostic data includes, but is not limited to, ECG signals (e.g., 12-lead or less than 12 lead signals, signals recorded on a portable/wearable device, or any other suitable ECG signal format), images of ECGs, cardiac imaging (e.g., echocardiographic (TTE) videos, point-of-care cardiac ultrasound, cardiac magnetic resonance imaging, or any other suitable cardiac imaging), or a combination thereof. In some embodiments, the method includes training the machine-learning model with a unimodal training dataset. In some embodiments, the method includes training the machine-learning model with a multimodal training dataset.

In some embodiments, the method includes pre-processing the cardiac diagnostic data prior to training. For example, in some embodiments, pre-processing the cardiac imaging includes loading pixel data; feeding randomly sampled frames through a convolutional neural network (CNN), the CNN classifying the frames by assigning a probability that a given video corresponds to a standard anatomical view; assigning a predicted view according to the highest assigned probability; cleaning and de-identifying the frames; augmenting the data; training a binary, video-level classifier to detect the presence of cardiomyopathy from controls; and normalizing intensities of each video clip.

In some embodiments, the machine-learning model includes a deep learning model. Other machine-learning models include, but are not limited to, transformer algorithms, convolutional neural networks or other specific types of deep learning models, as well as combinations of deep learning models with extreme gradient boosting, random forest and related machine learning or statistical modelling approaches. In some embodiments, the method includes training the machine-learning model to identify key patterns of cardiomyopathy. In some embodiments, the method includes training the machine-learning model to track longitudinal changes in the probability of cardiomyopathy among patients. In some embodiments, the method includes training the machine-learning model to detect subclinical cardiomyopathy. In some embodiments, the method includes training the machine-learning model to output a probability of a subject developing cardiomyopathy. Accordingly, the machine-learning models trained according to one or more of the embodiments disclosed herein form algorithms that track the evolution and subclinical progression of various forms of cardiomyopathies.

Also provided herein are methods of tracking, providing the likelihood of developing, and/or detecting cardiomyopathy in a subject. In some embodiments, the method includes providing cardiac diagnostic data from the subject, inputting the cardiac diagnostic data from the subject to the machine-learning model trained according to one of more of the embodiments disclosed herein, and tracking, determining the likelihood of developing, and/or detecting cardiomyopathy in a subject based upon the output from the machine-learning model. The cardiac diagnostic data data may be unimodal or multimodal, and can include any of the cardiac imaging and signal data disclosed herein. Similarly, the cardiomyopathy may include any of the cardiomyopathies disclosed herein (e.g., ATTR-CM).

The machine-learning models trained according to one or more of the embodiments disclosed herein can assess the presence of subtle ECG or echocardiographic signatures reflective of early cardiomyopathy, which can then be used to predict the rate of subclinical progression all the way to clinical disease and its eventual diagnosis, effectively discriminating individuals with early pre-clinical disease and risk stratifying their expected progression. For example, in some embodiments, following the inputting of the data, the machine-learning model outputs a probability of the subject developing cardiomyopathy based upon the ECG data. Additionally or alternatively, in some embodiments, the method includes determining that the subject has cardiomyopathy based upon the output from the machine-learning model. In some embodiments, the cardiomyopathy is pre-clinical cardiomyopathy, or detected during pre-symptomatic stages of cardiomyopathy. In some embodiments, the method includes providing personalized projection of disease trajectory, as opposed to simply identifying the cross-sectional presence of cardiomyopathy.

In some embodiments, the methods disclosed herein further include administering a treatment to the subject based upon the output of the machine-learning model. In some embodiments, the treatments may be administered earlier in the progression of cardiomyopathy and/or more accurate/targeted treatments may be administered as compared to existing methods due to the earlier, more accurate, and/or more definitive determinations of cardiomyopathy in subjects. Accordingly, in some embodiments, the methods disclosed herein reduce or eliminate rapid disease progression as a result of overlooked and/or under-diagnosed cardiomyopathy.

Without wishing to be bound by theory, it is believed that the methods disclosed herein demonstrate a unique and previously unreported role for artificial intelligence (AI)-enhanced interpretation of objective clinical data to define signatures of subclinical disease progression that can be used to identify such individuals during early stages and more accurately prognosticate their projected disease course to refine their management and deployment of novel therapeutics.

Also provided herein is an apparatus for detecting cardiomyopathy in a subject. In some embodiments, the apparatus includes a processor; a memory unit; and a communication interface. The processor is connected to the memory unit and the communication interface; and the processor and memory are configured to implement the method according to any of the embodiments disclosed herein.

Further provided herein is a computer readable storage medium storing computer-executable instructions for performing the method according to any of the embodiments disclosed herein.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

What is claimed is:

1. A method of training a machine-learning model to detect cardiomyopathy in a subject, the method comprising:
providing a training dataset, the training dataset including cardiac diagnostic data from a group of subjects;
identifying, in the training dataset, cardiac diagnostic data from positive subjects with cardiomyopathy and cardiac diagnostic data from control subject without cardiomyopathy; and
training the machine-learning model with the training dataset to discriminate between the cardiac diagnostic data from positive subjects and the cardiac diagnostic data from control subjects.

2. The method of claim 1, wherein the cardiac diagnostic data includes electrocardiographic (ECG) signals, images of ECGs, echocardiographic imaging, cardiac magnetic resonance imaging, nuclear cardiology examinations, or a combination thereof.

3. The method of claim 2, further comprising pre-processing the echocardiographic imaging, the pre-processing of the echocardiographic imaging including:
loading pixel data;
feeding randomly sampled images and/or frames through a convolutional neural network (CNN), the CNN classifying the frames by assigning a probability that a given video corresponds to a standard anatomical view;
assigning a predicted view according to the highest assigned probability;
cleaning and de-identifying the frames;
augmenting the data;
training a binary, video-level classifier to detect the presence of cardiomyopathy from controls; and
normalizing intensities of each video clip.

4. The method of claim 1, wherein the control subjects are at least one of age-matched to the positive subjects, sex-matched to the positive subjects, matching based on clinical and biological measurements of cardiac remodeling, and a combination thereof.

5. The method of claim 1, wherein, following the training step, the machine-learning model is configured to identify key patterns of cardiomyopathy.

6. The method of claim 1, wherein, following the training step, the machine-learning model is configured to track longitudinal changes in the probability of cardiomyopathy among patients.

7. The method of claim 1, wherein, following the training step, the machine-learning model is configured to detect subclinical cardiomyopathy.

8. The method of claim 1, wherein, following the training step, the machine-learning model is configured to output a probability of a subject developing cardiomyopathy.

9. The method of claim 1, wherein the cardiomyopathy comprises transthyretin amyloid cardiomyopathy (ATTR-CM).

10. The method of claim 9, wherein the subject was determined to be positive for ATTR-CM through an abnormal bone scintigraphy study or cardiac magnetic resonance imaging.

11. A method of detecting cardiomyopathy in a subject, the method comprising:
providing cardiac diagnostic data from the subject; and
inputting the cardiac diagnostic data from the subject to the machine-learning model trained according to claim 1;
wherein the machine-learning model outputs a probability of the subject developing cardiomyopathy based upon the cardiac diagnostic data.

12. The method of claim 11, wherein the ECG data is unimodal.

13. The method of claim 11, wherein the ECG data is multimodal.

14. The method of claim 11, wherein the cardiomyopathy includes restrictive or infiltrative cardiomyopathies with long indolent preclinical course, or combinations thereof.

15. The method of claim 14, wherein the cardiomyopathies with long indolent preclinical course include amyloid cardiomyopathy, hypertrophic cardiomyopathy, sarcoid cardiomyopathy, or a combination thereof.

16. The method of claim 11, further comprising determining that the subject has cardiomyopathy based upon the output from the machine-learning model.

17. The method of claim 16, wherein the cardiomyopathy is pre-clinical cardiomyopathy.

18. The method of claim 17, further comprising guiding the subject's eligibility for risk modifying therapies to reduce their risk of progression.

19. The method of claim 18, further comprising tracking a response to disease-modifying therapies in progressive cardiomyopathies using output probabilities of the model.

20. The method of claim 18, further comprising determining the subject's eligibility for use of a disease-modifying therapy or inclusion in a clinical study or clinical trial using output probabilities of the model.

21. An apparatus for detecting cardiomyopathy in a subject, the apparatus comprising:
a processor;
a memory unit; and
a communication interface;
wherein the processor is connected to the memory unit and the communication interface; and
wherein the processor and memory are configured to implement the method of claim 11.

22. A computer readable storage medium storing computer-executable instructions for performing the method of claim 11.
